(19) United States
(12) Patent Application Publication
(10) Pub. No.: US 2015/0018661 A1
(43) Pub. Date: Jan. 15, 2015

(54) NON-CONTACT SYSTEM FOR MEASURING CORNEAL PROPERTIES AND METHOD FOR MEASURING CORNEAL ELASTIC CONSTANT AND VISCOSITY CONSTANT

(71) Applicant: National Taiwan University, Taipei City (TW)

(72) Inventors: Jia-Yush YEN, Taipei City (TW); I-Jong WANG, Taipei City (TW); Chun-Ju HUANG, Taipei City (TW); Pei-Yi CHOU, Taipei City (TW)

(73) Assignee: National Taiwan University, Taipei City (TW)

(21) Appl. No.: 14/153,610

(22) Filed: Jan. 13, 2014

(30) Foreign Application Priority Data
Jul. 9, 2013 (TW) 102124535

(51) Int. Cl. A61B 3/16; A61B 3/10

(52) U.S. Cl. CPC A61B 3/165; A61B 3/1005
USPC 600/401

(57) ABSTRACT

A method for measuring corneal elastic constant and viscosity constant comprises steps of: ejecting compressed air toward a cornea of a live eye ball and measuring air pressure thereof; emitting infrared rays during an air ejecting period, for measuring corneal deformation caused by the compressed air applied to the cornea; and calculating an elastic constant and a viscosity constant of the cornea based on Kelvin-Voigt model by utilizing the corneal deformation measured via the infrared rays and the measured air pressure during the air ejecting period. One advantage of the present invention is to aid preliminary detection in eye diseases.

### CROSS-REFERENCE TO RELATED APPLICATIONS

This application claims priority to Taiwan Patent Application No. 102124535, filed on Jul. 9, 2013.

### TECHNICAL FIELD OF THE INVENTION

The present invention relates to an measurement of corneal properties of a live eye ball, and more particularly, to a non-contact system for measuring corneal properties and a method for measuring corneal elastic constant and viscosity constant.

### BACKGROUND OF THE INVENTION

A tonometer is currently used to measure ocular pressure. The measurement of ocular pressure aids preliminary detection for eye diseases (e.g., glaucoma). Generally speaking, the tonometer can be approximately classified into a contact type and a non-contact type. The contact-type tonometer needs to directly contact the eye ball of a participant. This type of tonometer is mainly divided into an applanation one and an indentation one. The applanation tonometer measures a necessary force applied to a small fixed area on the cornea to make the cornea flat so as to obtain the ocular pressure. The indentation tonometer measures the corneal deformation after applying a specific amount of force to the cornea.

The non-contact tonometer needs not to contact the eye ball of a participant mechanically. Accordingly, the non-contact tonometer is not harmful to the participant's eye and is not dangerous. An air-puff tonometer is one type of non-contact tonometer widely used in clinics. The air-puff tonometer makes the cornea deform by ejecting an air jet to the cornea, and measures the required time making the cornea reach a specific amount of deformation so as to obtain the ocular pressure.

The current ocular pressure measurements are all achieved by measuring the cornea, and thus these measurements are affected by central corneal thickness (CCT), corneal curvature (K), and corneal biomechanical properties. Accordingly, measuring properties of the cornea is also able to correct the measured ocular pressure moderately. In addition, the corneal properties also can be provided for an eye doctor for understanding the health condition of eye.

For example, an ocular response analyzer (ORA) is a new type of tonometer developed from the traditional air-puff tonometer. The ocular response analyzer can measure corneal hysteresis, and further confirm that more stiff a cornea, more close the relation between the central corneal thickness and the ocular pressure.

There are clinical studies on the meaning of corneal parameters measured by the ocular response analyzer. However, the conventional air-puff tonometer still can not define elastic constant and viscosity constant of the cornea, and there is no literature revealing these two parameters used in clinical applications. Accordingly, the present invention is to solve the problem of unable to measure the corneal elastic constant and viscosity constant by using the conventional air-puff tonometer.

### SUMMARY OF THE INVENTION

An objective of the present invention is to provide a non-contact system for measuring corneal properties and a method for measuring corneal elastic constant and viscosity constant, for obtaining corneal elastic constant and viscosity constant of a human eye.

To achieve the above objective, the present invention provides a non-contact system for measuring corneal properties, which comprises an air-puff device, ejecting compressed air toward a cornea of a live eye ball and measuring air pressure thereof; an infrared ray measuring device, emitting infrared rays during the air-puff device ejects the compressed air, for measuring corneal deformation caused by the compressed air applied to the cornea; and a processing unit, calculating an elastic constant and a viscosity constant of the cornea based on Kelvin-Voigt model by utilizing the corneal deformation measured by the infrared ray measuring device and the measured air pressure during the air-puff device ejects the compressed air.

In another aspect, the present invention provides a method for measuring corneal elastic constant and viscosity constant, comprising steps of: ejecting compressed air toward a cornea of a live eye ball and measuring air pressure thereof; emitting infrared rays during an air ejecting period, for measuring corneal deformation caused by the compressed air applied to the cornea; and calculating an elastic constant and a viscosity constant of the cornea based on Kelvin-Voigt model by utilizing the corneal deformation measured via the infrared rays and the measured air pressure during the air ejecting period.

The corneal elastic constant and viscosity constant measured in the present invention can be provided for an eye doctor to study the relation between any particular kind of eye disease and these two parameters so as to determine whether a man suffers from a specific type of eye disease. In another aspect, by improving the arithmetic unit or burning new algorithm, the non-contact system for measuring corneal properties and the method for measuring corneal elastic constant and viscosity constant as provided in the present invention are applicable to the widely-used air-puff tonometer and the ocular response analyzer in the market. Accordingly, the present invention can ease a burden of purchasing additional tonometer.

### BRIEF DESCRIPTION OF THE DRAWINGS

FIG. 1 is a schematic diagram showing a non-contact system for measuring corneal properties implemented according to the present invention.

FIG. 2 is a schematic diagram showing corneal deformation in various of stages due to pressure applied by an air jet.

FIG. 3 is a diagram showing measured air pressure and infrared signal intensity varying with time during measuring corneal properties of a participant.

FIG. 4 is a schematic diagram illustrating Kelvin-Voigt model.

FIG. 5 is a schematic diagram showing an instantaneous rate of signal change used in calculating corneal viscosity constant.

FIG. 6 is a schematic diagram showing an average slope used in calculating corneal viscosity constant.

FIG. 7 is a flow chart of a method for measuring corneal elastic constant and viscosity constant implemented according to the present invention.

### DETAILED DESCRIPTION OF THE INVENTION

The present invention provides a system and method for measuring corneal elastic constant and viscosity constant for a doctor to do clinical researches to find correlation between any particular kind of eye disease and these two parameters of the cornea.

For a patient with some type of eye disease (e.g., keratoconus), an eye doctor can utilize the measuring system and method provided in the present invention to measure the elastic constant and viscosity constant of the patient's cornea and study the relation between this type of eye disease and theses parameters. This benefits preliminary detection in eye diseases greatly.

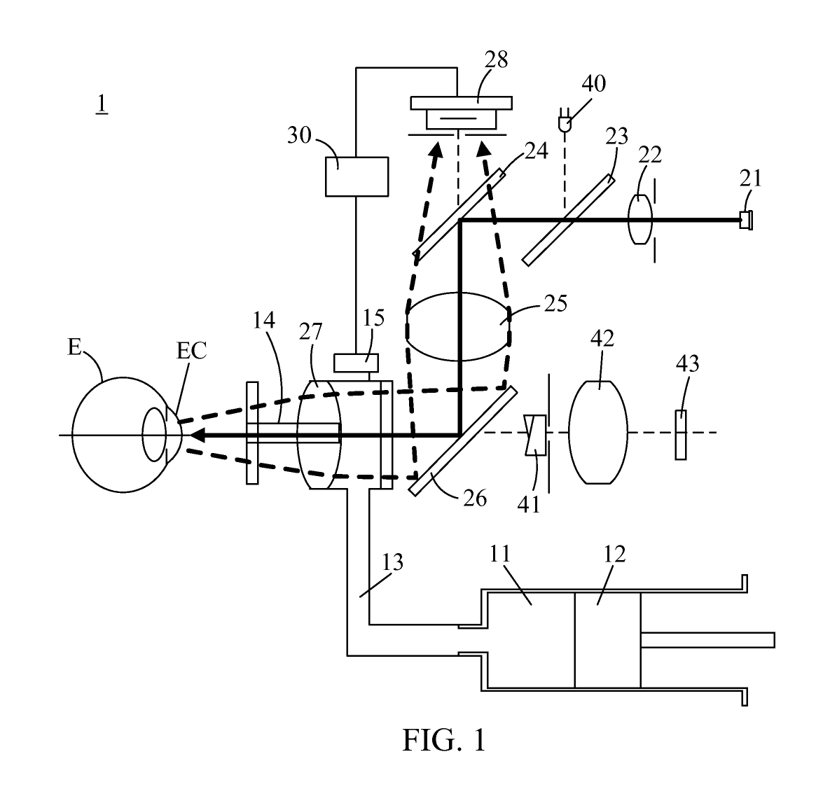

FIG. 1 is a schematic diagram showing a non-contact system for measuring corneal properties implemented according to the present invention. The measuring system 1 is suitable for measuring the properties of a cornea EC of a live eye ball E. The measuring system 1 is an air-puff measuring system, which may be incorporated with ocular pressure measurement so as to become an air-puff tonometer. The measuring system 1 of the present invention mainly comprises an air-puff device, an infrared ray measuring device, and an arithmetic unit. The arithmetic unit is able to calculate the elastic constant and viscosity constant of the cornea EC according to the air pressure measured by the air-puff device and infrared signal distribution measured by the infrared ray measuring device.

Please further refer to FIG. 1. The air-puff device mainly comprises a cylinder 11, a piston 12, a nozzle 14, and an air-pressure gauge 15. During measuring properties of the cornea, the piston 12 compresses the air in the cylinder 11, the compressed air is ejected from the nozzle 14 via a pipeline 13, and the ejected air presses the cornea of the eye ball such that the cornea deforms. The air-pressure gauge 15 measures and records the air pressure in the chamber during the process. The air pressure measured by the air-pressure gauge 15 is approximate to the pressure endured by the cornea.

The infrared ray measuring device mainly comprises an infrared source 21, a plurality of optical path adjuster (e.g., a beam splitter, a lens, and etc.), and a sensor 28. As shown in FIG. 1, during the air-puff device ejects the compressed air for measuring the corneal properties, the infrared source 21 emits infrared rays, which projects to the cornea of the eye ball after passing a lens 22, beam splitters 23, 24, a lens 25, a beam splitter 26, and a lens 27 in order (the optical path indicated by a heavy solid line in FIG. 1). After reflected by the cornea, the infrared rays pass the lens 27, the beam splitter 26, the lens 25, and the beam splitter 24 in order and then are received by the sensor 28 (the optical path indicated by a heavy dotted line in FIG. 1). When the cornea deforms due to the compressed air ejected from the nozzle 14, the ratio or amount of reflected infrared rays is changed. Accordingly, the intensity of infrared signals measured by the sensor 28 is varied in the measuring process.

The light rays emitted form a light emitting diode (LED) 40 follows an optical path formed by passing the beam splitters 23, 24, the lens 25, the beam splitter 26, and the lens 27 and then projects a measuring light spot on the cornea. The measuring light spot can be observed by an operating personnel through an optical path formed by passing the lens 27, the beam splitter 26, an adjusting prism 41, a lens 42, and a viewing window 43. Meanwhile, the operating personnel can view the cornea at an appropriate angle and position by adjusting the adjusting prism 41.

The arithmetic unit (i.e., a processing unit 30) receives the air pressure measured by the air-pressure gauge 15 of the air-puff device during the air ejecting period and the infrared signals measured by the sensor 28 of the infrared ray measuring device, and then calculates the elastic constant and viscosity constant of the cornea according to the measured air pressure and the corneal deformation indicated by the measured infrared signals. Further, the air-pressure gauge 15 may output a diagram indicating the air pressure varying with time, and the sensor 28 may output a diagram indicating the infrared signal intensity varying with time. The processing unit 30 is able to calculate the elastic constant and viscosity constant of the cornea based on Kelvin-Voigt model and the distribution of the air pressure and the infrared signal intensity, and this will be described later.

The system and method for measuring corneal elastic constant and viscosity constant as provided in the present invention is applicable to the widely-used air-puff tonometer, and to an ocular response analyzer (ORA) which provides a further improvement on such type of tonometer.

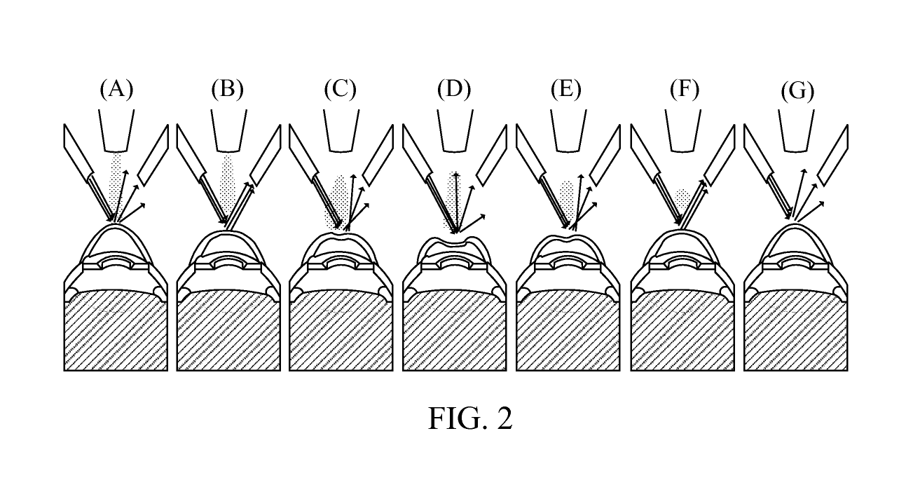

Referring to FIG. 2, the infrared rays also can be incident on the cornea from the sides. After the infrared rays are reflected by the cornea, the reflected infrared rays are received at an opposite side. During measuring corneal properties of some particular participant, the corneal is convex in the beginning (as shown in Part (A) of FIG. 2), and then the applied pressure from the ejected gas makes the cornea become flat such that the cornea is at a first flat position, and meanwhile the sensor receives high intensity of infrared rays (as shown in Part (B)). The ejected gas continuously presses the cornea such that the cornea becomes concave (as shown in Parts (C) and (D)). At the time the cornea is in a most concave state, the sensor receives few infrared rays since its focus near the cornea. Next, the pressure of the ejected gas declines, the cornea backs to the flat state from the most concave state such that the cornea is at a second flat position (as shown in Parts (E) and (F)), and meanwhile the sensor receives high intensity of infrared rays again. Finally, when the cornea is reverted to convex, the infrared rays are scattered and the amount of infrared rays received by the sensor decreases accordingly.

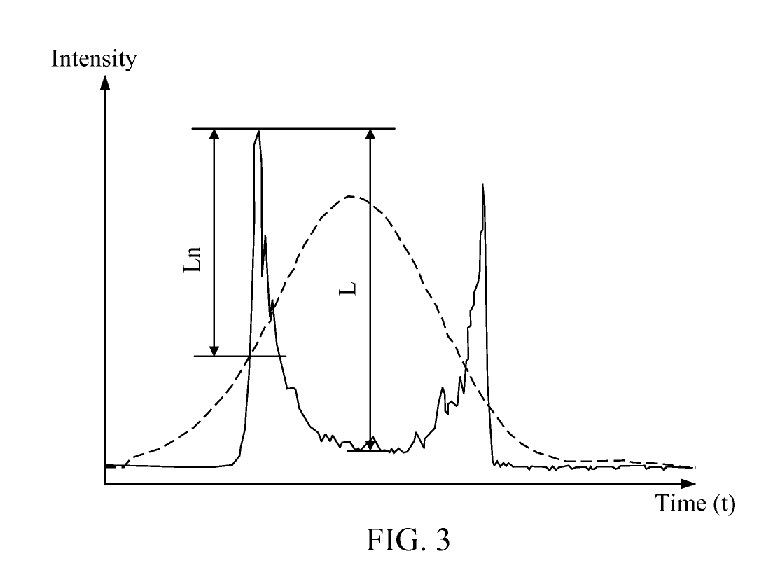

FIG. 3 is a diagram showing the measured air pressure and infrared signal intensity varying with time during measuring corneal properties of a participant. In FIG. 3, the dotted line represents a pressure curve measured by the air-pressure gauge and the solid line represents intensity distribution of infrared signals measured by the sensor. Please refer to FIG. 2 and FIG. 3. The pressure curve approximates to Gaussian distribution curve. When the cornea is at the first flat position, a first signal peak (at left side of FIG. 3) appears on the infrared signal distribution. When the cornea is at the second flat position, a second signal peak (at right side of FIG. 3) appears on the infrared signal distribution. When the cornea is in the most concave state, the air pressure at this time nears a maximum and the infrared signal intensity apparently decreases, as a minimum.

In the present invention, the calculation of elastic constant and viscosity constant of the cornea will be detailed as follows.

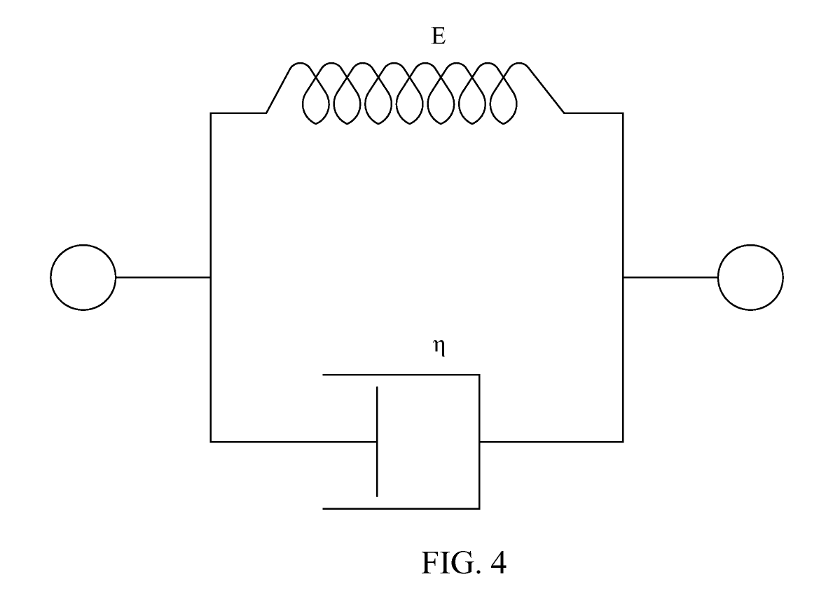

The corneal elasticity and viscosity can be described by using Kelvin-Voigt model (see FIG. 4), as represented by the following Equation (1):

$$σ ( t ) = E ɛ ( t ) + η \frac{d ɛ}{d t}$$

where σ is a stress endured by the cornea, ε is a strain of the cornea, t represents time, E is the elastic constant, and η is the viscosity constant.

The stress endured by the cornea can be represented by the air pressure measured by the air-pressure gauge and the strain of the cornea can be estimated approximately by utilizing the infrared signal intensity measured by the sensor. Referring to FIG. 3, the strain of the cornea ε may be defined as ε=ΔL/L, where L is the difference between a maximum signal value and a minimum signal value among two signal peaks on the infrared signal distribution, which is defined as L=Lₘₐₓ−Lₘᵢₙ, and ΔL is an amount of deformation at a specific moment, which is defined as ΔL=Lₘₐₓ−L_{n, where L}ₙ is a signal value at that moment.

I. Calculation of Elastic Constant E:

When the cornea is in the most concave state, the amount of corneal deformation is a maximum. Meanwhile, the infrared signal curve becomes flat and dε/dt is zero. Accordingly, the elastic constant E of the cornea can be calculated by the following Equation (2):

$$E = \frac{σ ( {t}_{E} )}{ɛ ( {t}_{E} )}$$

where t_{E} is a moment that dε/dt is zero;

That is, the elastic constant E can be calculated by utilizing the air pressure and the strain at the moment t_{E} the signal intensity curve becomes flat among the two signal peaks.

II. Calculation of Viscosity Constant η:

When the cornea deforms initially or is reverted to convex, the strain of the cornea is zero, i.e., ε(t)=0, and the viscosity constant η of the cornea can be calculated by the following Equation (3):

$$η = \frac{σ ( {t}_{η} )}{d ɛ / d {t}_{η}}$$

where t_{η} is a moment that ε(t) is zero.

The differential value dε/dt of the strain at the moment the cornea deforms initially (or is reverted to convex) is approximate to the value where the cornea is at the first flat position (or the second flat position). Accordingly, the differential value of the infrared signal intensity at the moment the cornea is at the flat positions can be used to represent the differential value at this period. The stress endured by the cornea at this period can be represented by the air pressure at the moment the infrared signal intensity is dramatically changed. Accordingly, the viscosity constant η can be calculated by the above Equation (3).

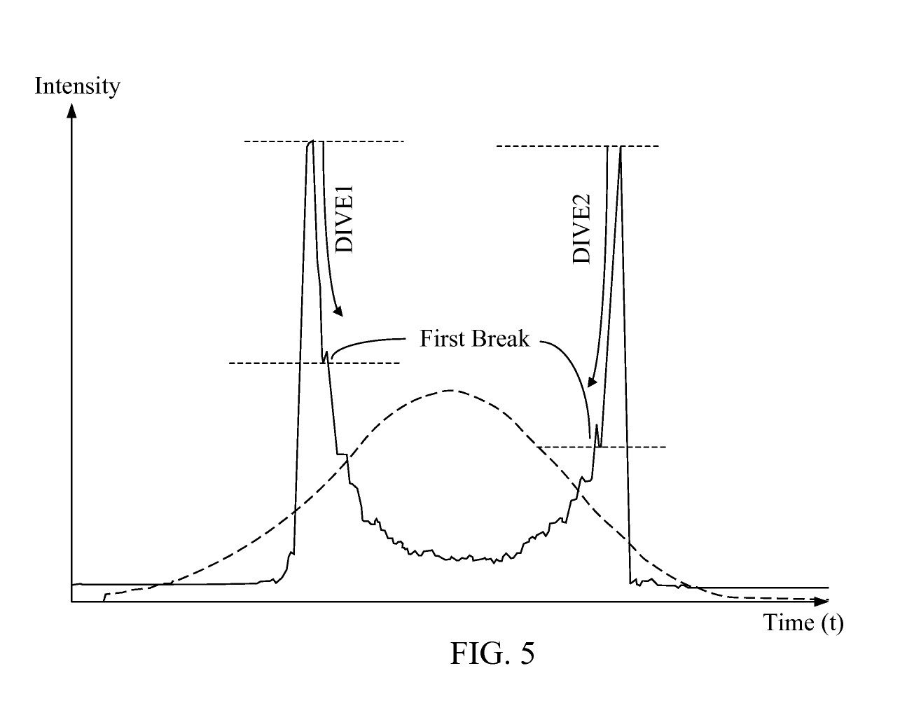

In one embodiment, the differential value dε/dt of this period can be obtained by calculating an instantaneous rate of signal change at the moment the cornea is at the first flat position (or the second flat position). For example, the value DIVE1 (or DIVE2) given by the ocular response analyzer can be used to represent the instantaneous rate of signal change, as shown in FIG. 5. The values DIVE1 and DIVE2 are slopes measuring from the signal peak value to a first break point (i.e., first break of signal).

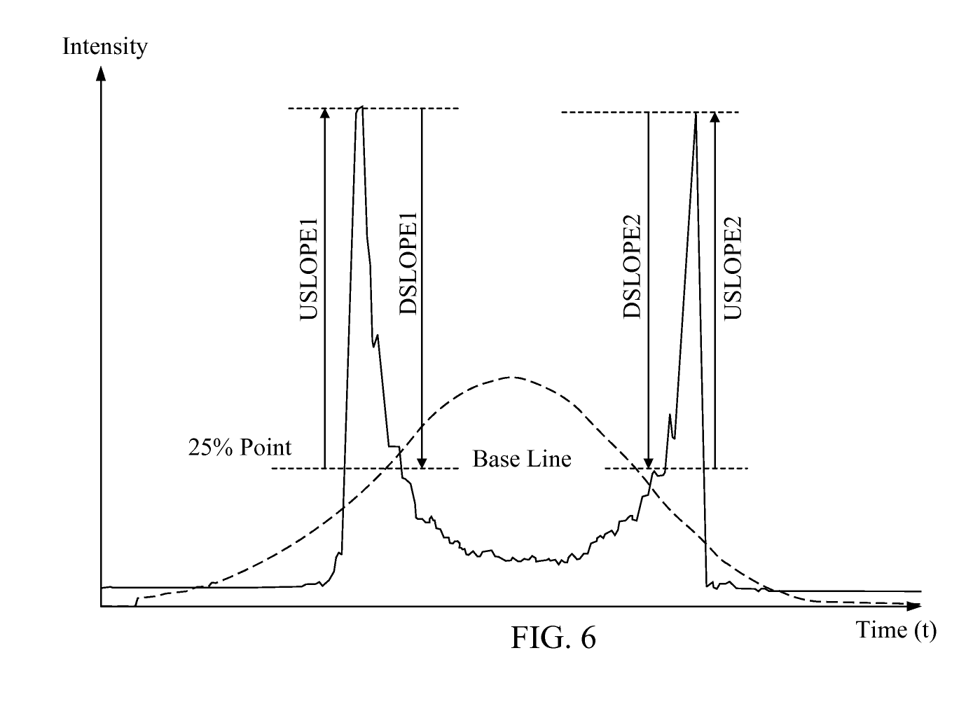

In another embodiment, the differential value dε/dt of this period can be obtained by calculating an average slope measuring from the signal peak value (i.e., at the moment the cornea is at the first flat position (or the second flat position)) to a specific base point. For example, the values USLOPE1 and DSLOPE1 (alternatively, USLOPE2 and DSLOPE2) given by the ocular response analyzer can be used to represent the average slope, as shown in FIG. 6. The values USLOPE1, DSLOPE1, USLOPE2, and DSLOPE2 are slopes measuring from the signal peak value to a base point having 25% intensity of the signal peak.

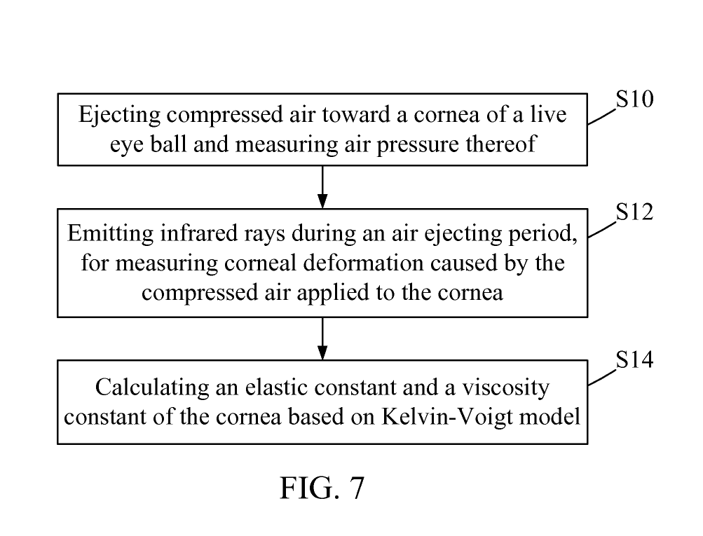

Referring to FIG. 7, the present invention provides a method for measuring corneal elastic constant and viscosity constant, said method comprises following steps.

In Step S10, compressed air is ejected toward a cornea of a live eye ball and corresponding air pressure is measured. As shown in FIG. 1, the piston 12 compresses the air or gas in the cylinder 11 and the compressed air is ejected from the nozzle 14. The ejected air or fluid presses a cornea EC of a live eye ball E such that the cornea EC deforms. The air-pressure gauge 15 measures and records the air pressure in the chamber during the process, and then obtains a diagram indicating the measured air pressure varying with time.

In Step S12, infrared rays are emitted during an air ejecting period in Step S10, for measuring corneal deformation caused by the compressed air applied to the cornea. As shown in FIG. 1, the infrared source 21 emits the infrared rays. After reflected by the cornea EC, the infrared rays are received by the sensor 28. The intensity of infrared signals measured by the sensor 28 is varied with time due to the corneal deformation. A diagram indicating the infrared signal intensity varying with time is obtained.

In Step S14, the elastic constant and viscosity constant of the cornea are calculated by utilizing the above-mentioned Kelvin-Voigt model. In this step, in two extreme conditions of the corneal deformation (i.e., maximum deformation and zero deformation), the elastic constant and viscosity constant of the cornea are respectively calculated by utilizing above-mentioned Equations (2) and (3), based on the diagram indicating the measured air pressure and infrared signal intensity varying with time, obtained from measuring properties of a participant's cornea.

The corneal elastic constant and viscosity constant measured in the present invention can be provided for an eye doctor to study the relation between any particular kind of eye disease and these two parameters so as to determine whether a man suffers from a specific type of eye disease. In another aspect, by improving the arithmetic unit or burning new algorithm, the non-contact system for measuring corneal properties and the method for measuring corneal elastic constant and viscosity constant as provided in the present invention are applicable to the widely-used air-puff tonometer and the ocular response analyzer in the market. Accordingly, the present invention can ease a burden of purchasing additional tonometer.

While the preferred embodiments of the present invention have been illustrated and described in detail, various modifications and alterations can be made by persons skilled in this art. The embodiment of the present invention is therefore described in an illustrative but not restrictive sense. It is intended that the present invention should not be limited to the particular forms as illustrated, and that all modifications and alterations which maintain the spirit and realm of the present invention are within the scope as defined in the appended claims.

What is claimed is:

1. A non-contact system for measuring corneal properties, comprising:
an air-puff device, ejecting compressed air toward a cornea of a live eye ball and measuring air pressure thereof;
an infrared ray measuring device, emitting infrared rays during the air-puff device ejects the compressed air, for measuring corneal deformation caused by the compressed air applied to the cornea; and
a processing unit, calculating an elastic constant and a viscosity constant of the cornea based on Kelvin-Voigt model by utilizing the corneal deformation measured by the infrared ray measuring device and the measured air pressure during the air-puff device ejects the compressed air.

2. The non-contact system for measuring corneal properties according to claim 1, wherein the processing unit calculates the elastic constant and the viscosity constant respectively in two extreme conditions of the corneal deformation.

3. The non-contact system for measuring corneal properties according to claim 2, wherein the Kelvin-Voigt mode is represented by:
$$σ ( t ) = E ɛ ( t ) + η \frac{d ɛ}{d t}$$
where σ is a stress endured by the cornea, ε is a strain of the cornea, t represents time, E is the elastic constant, and η is the viscosity constant;
wherein when deformation amount of the cornea is a maximum, dε/dt is zero, and the elastic constant is calculated by:
$$E = \frac{σ ( {t}_{E} )}{ɛ ( {t}_{E} )}$$
where t_{E} is a moment that dε/dt is zero;
wherein when the cornea deforms initially, the strain is zero, ε(t)=0, and the viscosity constant is calculated by:
$$η = \frac{σ ( {t}_{η} )}{d ɛ / d {t}_{η}}$$
where t_{η} is a moment that ε(t) is zero.

4. The non-contact system for measuring corneal properties according to claim 3, wherein an instantaneous rate of change on a signal peak of infrared signals measured by the infrared ray measuring device is served as a value of dε/dt_{η} in calculating the viscosity constant.

5. The non-contact system for measuring corneal properties according to claim 3, wherein an average slope on a signal peak of infrared signals measured by the infrared ray measuring device is served as a value of dε/dt_{η} in calculating the viscosity constant.

6. A method for measuring corneal elastic constant and viscosity constant, comprising steps of:
ejecting compressed air toward a cornea of a live eye ball and measuring air pressure thereof;
emitting infrared rays during an air ejecting period, for measuring corneal deformation caused by the compressed air applied to the cornea; and
calculating an elastic constant and a viscosity constant of the cornea based on Kelvin-Voigt model by utilizing the corneal deformation measured via the infrared rays and the measured air pressure during the air ejecting period.

7. The method for measuring corneal elastic constant and viscosity constant according to claim 6, wherein the elastic constant and the viscosity constant are calculated respectively in two extreme conditions of the corneal deformation.

8. The method for measuring corneal elastic constant and viscosity constant according to claim 7, wherein the Kelvin-Voigt mode is represented by:
$$σ ( t ) = E ɛ ( t ) + η \frac{d ɛ}{d t}$$
where σ is a stress endured by the cornea, ε is a strain of the cornea, t represents time, E is the elastic constant, and η is the viscosity constant;
wherein when deformation amount of the cornea is a maximum, dε/dt is zero, and the elastic constant is calculated by:
$$E = \frac{σ ( {t}_{E} )}{ɛ ( {t}_{E} )}$$
where t_{E} is a moment that dε/dt is zero;
wherein when the cornea deforms initially, the strain is zero, ε(t)=0, and the viscosity constant is calculated by:
$$η = \frac{σ ( {t}_{η} )}{d ɛ / d {t}_{η}}$$
where t_{η} is a moment that ε(t) is zero.

9. The method for measuring corneal elastic constant and viscosity constant according to claim 8, wherein an instantaneous rate of change on a signal peak of measured infrared signals is served as a value of dε/dt_{η} in calculating the viscosity constant.

10. The method for measuring corneal elastic constant and viscosity constant according to claim 8, wherein an average slope on a signal peak of measured infrared signals is served as a value of dε/dt_{η} in calculating the viscosity constant.
